(19) United States
(12) Patent Application Publication
(10) Pub. No.: US 2007/0112368 A1
(43) Pub. Date: May 17, 2007

(54) METHOD FOR USING A TROCAR WITH INFLATABLE SEAL FOR TRANSSTOMAL ENDOSCOPIC PROCEDURES

(75) Inventor: Basel S. Hassoun, Oklahoma City, OK (US)

Correspondence Address: APPLIED MEDICAL RESOURCES CORPORATION, 22872 Avenida Empresa, Rancho Santa Margarita, CA, 92688, US

(73) Assignee: APPLIED MEDICAL RESOURCES CORPORATION, Rancho Santa Margarita, CA (US)

(21) Appl. No.: 11/561,181

(22) Filed: Nov. 17, 2006

Related U.S. Application Data
(60) Provisional application No. 60/597,229, filed on Nov. 17, 2005.

(51) Int. Cl. A61M 29/00

(52) U.S. Cl. 606/191

(57) ABSTRACT

A method for using a trocar assembly for transstomal endoscopic procedures. The method comprises the steps of providing a trocar comprising a handle and a tubular cannula extending from the handle, the handle defining an operating channel continuous with a lumen of the cannula, wherein an annular balloon is disposed on the outside of the cannula; inserting the distal end of the trocar cannula into the stoma and advancing the trocar until the annular balloon is inside the stoma; and inflating the annular balloon to sealingly engage the opening of the stoma.

This is a non-provisional application claiming the priority of provisional application Ser. No. 60/597,229, filed on Nov. 17, 2005, which is fully incorporated herein by reference.

### FIELD

The present invention relates generally to endoscopic tools and more particularly, but without limitation, to endoscopic trocar assemblies, and methods of using trocar assemblies for transstomal endoscopic procedures.

### BACKGROUND

Mechanical trocars typically include a cannula defining a working channel and a housing, which encloses valves that function to inhibit the escape of gas or fluids. Typically, the cannula of the trocar is adapted to be positioned across the abdominal wall of a patient using an obturator, which is initially inserted into the working channel and then removed once an entry site through the abdominal wall has been made and the cannula is in place. Various elongated instruments can be inserted through the working channel of the trocar to reach and perform operative functions at a site within the abdomen.

In most patients, the obturator creates an entry site into the body cavity through with the cannula of the trocar is inserted. In patients who have had a prior colostomy or iliostomy, however, the stoma provides a pre-existing entry site into the body for examination and/or treatment of intestinal or urological structures.

A stoma is an opening to the intestine on the abdominal wall, usually created by a surgeon. A stoma is necessary when parts of the normal intestine must be bypassed, for example when bowel surgery has removed important lengths of the intestine. There are several types of stoma, which take their name from the part of the intestine that forms the opening. These include gastrostomy, an opening from the skin directly into the stomach; jejunostomy, an opening from the first part of the small bowel, ileostomy, an opening from the small bowel, and colostomy, an opening from the large bowel. Another type of stoma, called a urostomy, is an artificial connection between the urinary tract (the kidneys, bladder and tubes that connect them) and the abdominal wall, sometimes referred to as a ‘urinary conduit’.

Transstomal endoscopic procedures may play an important role in the management of patients who have undergone procedures such as colonostomies. For example, at least one study has shown that transstomal endoscopic exploration in rectal cancer patients who have undergone a colonoscopy is a safe and effective tool in monitoring for recurrence of cancer, as rectal cancer patients are at high risk for metachronous large-bowel primaries. See, Zilli, L. Pietroiust, M. and Bertario, L. (1987) Dis. Colon Rectum 30(9): 687-691. However, endoscopic procedures on intestinal structures in patients with colostomies and on urological structures in patients with iliostomies are problematic because there is no sphincter in the stoma to contain the air or fluid in the colon or conduit typically introduced during endoscopic procedures. In addition, the trocar sleeve on the conventional trocar assembly is too long for effective use in a stoma. The present invention is directed to an apparatus and a method of conducting endoscopic procedures through a stoma, which address these problems.

### SUMMARY

The present invention comprises a trocar assembly for transstomal endoscopic procedures and a method for using the same. The assembly comprises a handle and a tubular trocar sleeve extending from the handle. The handle defines an operating channel continuous with the lumen of the sleeve. An annular balloon is disposed on the outside of the sleeve. Also included is an inflation system to connect the balloon with a source of fluid. The trocar may also include a seal near the opening to the operating channel in the handle for sealing the opening during use of the trocar.

The present invention also comprises a method for inserting a cannula through a stoma in the wall of a body cavity for use in transstomal endoscopy, comprising the steps of: providing a trocar comprising a handle and a tubular cannula extending from the handle, the handle defining an operating channel continuous with a lumen of the cannula, wherein an annular balloon is disposed on the outside of the cannula; inserting the distal end of the trocar cannula into the stoma and advancing the trocar until the annular balloon is inside the stoma; and inflating the annular balloon to sealingly engage the opening of the stoma.

### BRIEF DESCRIPTION OF THE DRAWINGS

FIG. 1 is a side elevational view of a trocar assembly constructed in accordance with the present invention. The balloon on the trocar sleeve is deflated. Only the tip of the introducer is shown.

FIG. 2 is a side elevational view of the trocar assembly of FIG. 1 with the balloon inflated.

FIG. 3 is a perspective view of the trocar assembly of FIG. 1.

### DETAILED DESCRIPTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the structures and/or methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

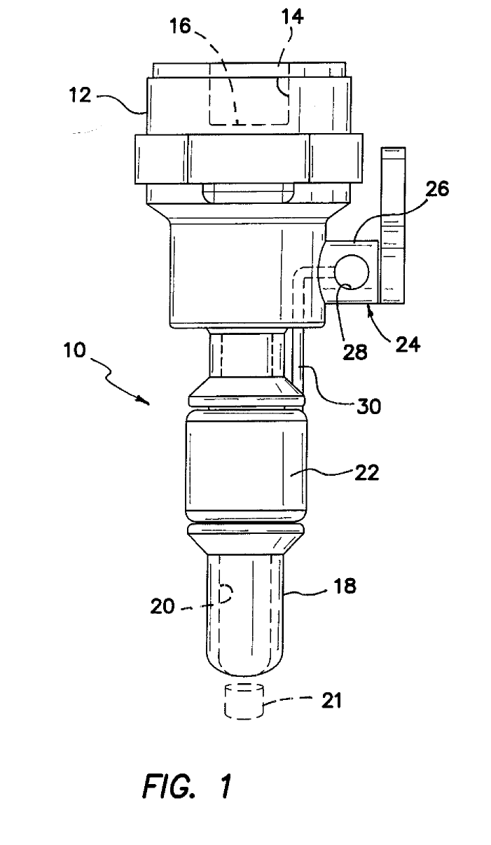

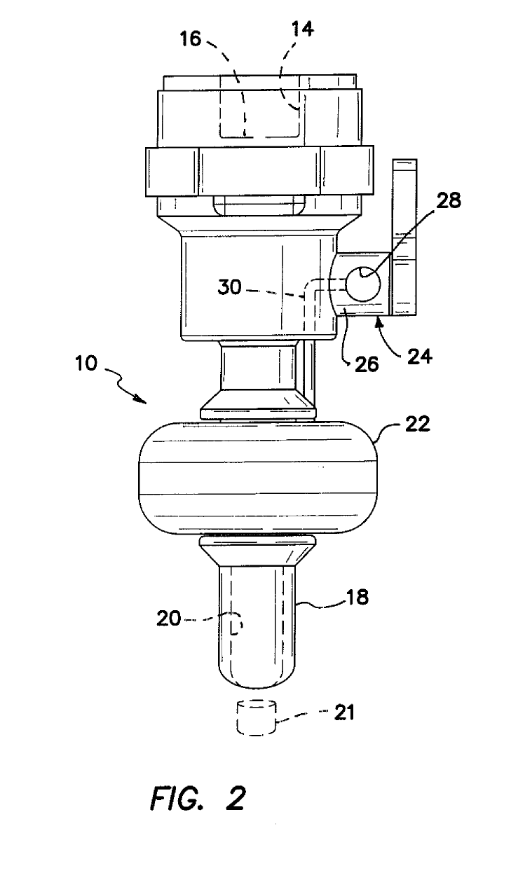

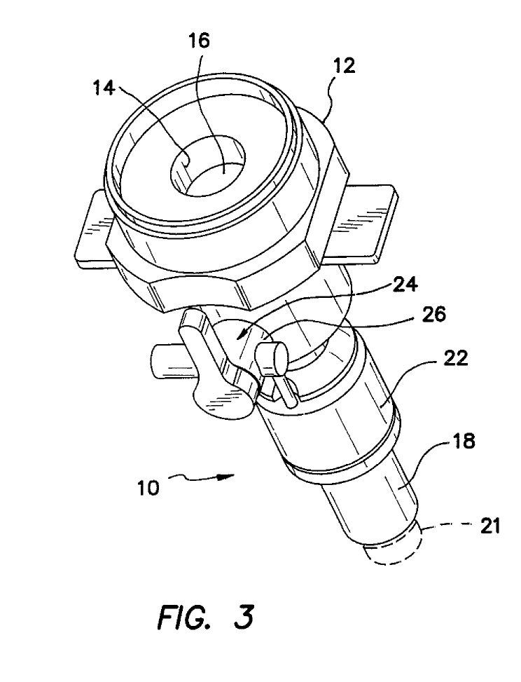

The present invention is directed to a trocar assembly for transstomal endoscopic procedures. A preferred embodiment of the inventive trocar assembly is shown in FIGS. 1-3, to which reference now is made.

The trocar assembly is designated generally by the reference numeral 10. The assembly 10 comprises a head or handle 12 by which the assembly 10 is controlled for atraumatic insertion of the trocar into the stoma. An operating channel 14 is provided for insertion of a colonoscope, cytoscope or other tools into the trocar. The opening into the operating channel 14 preferably includes a valve or seal, indicated at 16, for forming an air seal with the tool being inserted into the trocar.

A tubular cannula or sleeve 18 extends from the handle 12. The lumen 20 of the sleeve communicates with the operating channel 14 in the handle 12. The size and length of the sleeve 18 may vary. A preferred sleeve diameter for trocars used in patients having colostomies or iliostomies is a diameter of 19 mm, as this size will accommodate the easy introduction of most colonoscopes and cytoscopes. As one skilled in the art will readily appreciate, however, the diameter may be larger or smaller, depending on the type and size of the patient's stoma and the instruments to be used during the procedure.

The sleeve 18 is shorter than the sleeve on a conventional endoscopic trocar. In one embodiment, the sleeve of the present trocar is about two to three (3) inches in length, preferably about three inches. Again, however, this length will vary depending on the type and size of the stoma, the patient's anatomy, the particular procedure being undertaken, and the like.

The introducer used in the assembly 10 is of conventional design and, therefore, is not shown or described in detail. However, as will be understood from the drawings, the size of the introducer is adjusted to conform to the sleeve 18. In addition, the end of the introducer 21 preferably is blunted to minimize the risk of trauma to the tissues during insertion. Unlike obturators used in conventional trocars, the end of the introducer does not provide a cutting function to provide an entry site through the body wall but instead serves to open the stoma to facilitate passage of the trocar sleeve or cannula through the stoma. The introducer's smooth or blunt tip 21 helps prevent damage to the stoma or to the intestine during insertion of the trocar.

An annular balloon 22 is disposed on the sleeve 18. The balloon may be adhesively affixed to the sleeve and/or disposed between a pair of brackets 23 that prevent movement of the balloon along the trocar sleeve. The balloon 22 is sized and positioned to sealingly engage the stoma when the sleeve is inserted into the stoma and the balloon is inflated.

An inflation system 24 is included for connecting the balloon 22 to a source of fluid or gas, such as water or air. The inflation system 24 includes a valve 26 that may be mounted on the handle 12. The valve 24 includes a port 28 connected by a passageway 30 to the balloon 22. Preferably, the port 28 is adapted to receive the tip of a syringe (not shown). In this way, inflation and deflation of the balloon 22 is accomplished simply by injecting and withdrawing air (or other gas, or fluid) with a syringe.

Given the above-described structure, the use and operation of the trocar assembly of the present invention should be readily apparent to those of skill in this art. Briefly, an introducer is inserted into the operating channel of the trocar and through the lumen so that the blunt tip of the introducer protrudes slightly from the rounded distal end of the trocar sleeve. The sleeve 18 is inserted a distance into the colon or ileal conduit through the stoma and the introducer is removed. Preferably, the sleeve is inserted into the colon or conduit until the deflated balloon is just inside the stoma opening, inside the lumen of the colon or conduit. Next, using a syringe, air or water is injected into the balloon 22 so that the balloon engages the inner wall of the colon or conduit to seal the opening of the stoma.

With the trocar sleeve 18 positioned, the colonoscope or cytoscope or other tool is inserted into the opening into the operating channel 14 in the handle 12. Once the tool is inserted a sufficient distance, air, water or another suitable fluid is introduced into the conduit or colon. The seal 16 at the opening of the operating channel prevents air or fluids from escaping through the trocar 10, and the inflated balloon forms a seal between the intestinal wall or stoma and the trocar sleeve 18.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive. Also, all the examples provided throughout the entire description should be considered in all respects as illustrative and not restrictive. The scope of the invention is, therefore, indicated by the following claims rather than by the foregoing description. All changes, modifications, and variations coming within the meaning and range of equivalency of the claims are to be considered within their scope.

1. A method for introducing a trocar through a stoma in the wall of a body cavity for use in transstomal endoscopy, comprising the steps of:
providing a trocar comprising a handle and a tubular cannula extending from the handle, the handle defining an operating channel continuous with a lumen of the cannula, wherein an annular balloon is disposed on the outside of the cannula,
inserting the distal end of the trocar cannula into the stoma and advancing the trocar until the annular balloon is inside the stoma, and
inflating the annular balloon to sealingly engage the opening of the stoma

2. The method of claim 1, wherein the trocar further comprises an inflation system to connect the balloon with a source of fluid.

3. The method of claim 2, wherein the inflation system comprises a valve, a port and a passageway connecting the port to the annular balloon.

4. The method of claim 2, wherein the annular balloon is inflated by injecting a fluid into the inflation system.

5. The method of claim 1, wherein a syringe is used to inject a fluid or a gas into the annular balloon to thereby inflate it.

6. The method of claim 1, wherein the trocar further comprises a seal in the operating channel in the handle for sealing the operating channel during use of the trocar.

7. The method of claim 1, wherein the length of the trocar cannula is from about 2 inches to about 3 inches.

8. The method of claim 1, wherein the diameter of the trocar cannula is from about 15 mm to about 20 mm.

9. The method of claim 1, further comprising the step of inserting an introducer having a blunt end into the operating channel and through the trocar cannula until the blunt tip of the introducer projects from the distal end of the cannula, prior to inserting the trocar cannula into the stoma.

10. The method of claim 1 wherein the stoma is formed from the large bowel and the annular balloon is inflated to engage the interior wall of the large bowel at the site of the stoma.

11. The method of claim 10, further comprising the step of introducing a gas or fluid into the large bowel through the trocar.

12. The method of claim 1 wherein the stoma is formed from the small intestine and the annular balloon is inflated to engage the interior wall of the small intestine at the site of the stoma.

13. The method of claim 12, further comprising the step of introducing a gas or fluid into the small intestine through the trocar.
